(19) United States
(12) Patent Application Publication
(10) Pub. No.: US 2010/0303727 A1
(43) Pub. Date: Dec. 2, 2010

(54) OPTICAL IMAGING AGENTS

(76) Inventors: Alan Cuthertson, Oslo (NO); Edvin Wilhelm Johannesen, Oslo (NO); Michael Edward Cooper, Cardiff (GB)

Correspondence Address: GE HEALTHCARE BIO-SCIENCES CORP.;PATENT DEPARTMENT, 101 CARNEGIE CENTER, PRINCETON, NJ, 08540, US

(21) Appl. No.: 12/600,033

(22) PCT Filed: May 16, 2008
(86) PCT No.: PCT/GB2008/001693

§ 371 Date: Nov. 13, 2009

(30) Foreign Application Priority Data
May 16, 2007 (GB) 0709441.0
Aug. 13, 2007 (GB) 0715682.1
Aug. 20, 2007 (GB) 0716175.5

(51) Int. Cl. A61K 49/00; C07K 7/06; C07D 403/06

(52) U.S. Cl. 424/9.6; 530/328; 548/455

(57) ABSTRACT

The present invention relates to imaging agents suitable for in vivo optical imaging, which comprise conjugates of pentamethine cyanine dyes having reduced non-specific binding, e.g. to plasma proteins. This is achieved by control of the nature and location of the sulfonic acid substituents, in particular the sulfoalkyl groups. Also disclosed are pharmaceutical compositions and kits, as well as in vivo imaging methods.

### FIELD OF THE INVENTION

The present invention relates to imaging agents suitable for in vivo optical imaging, which comprise conjugates of pentamethine cyanine dyes having reduced non-specific binding, eg. to plasma proteins. This is achieved by control of the nature and location of the sulfonic acid substituents, in particular the sulfoalkyl groups. Also disclosed are pharmaceutical compositions and kits, as well as in vivo imaging methods.

### BACKGROUND TO THE INVENTION

U.S. Pat. No. 6,083,485 and counterparts discloses in vivo near-infrared (NIR) optical imaging methods using cyanine dyes having an octanol-water partition coefficient of 2.0 or less. Also disclosed are conjugates of said dyes with “biological detecting units” of molecular weight up to 30 kDa which bind to specific cell populations, or bind selectively to receptors, or accumulate in tissues or tumours. The dyes of U.S. Pat. No. 6,083,485 may also be conjugated to macromolecules, such as polylysine, dextran or polyethylene glycol. No specific dye-conjugates are disclosed.

WO 00/16810 discloses NIR fluorescent contrast agents which have 3 or more sulfonic acid groups in the molecule, and are of formula A:

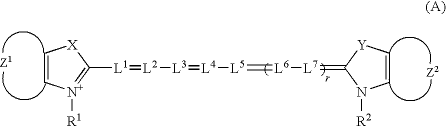

wherein:

R¹ and R² are the same or different and each is a substituted or unsubstituted alkyl;

Z¹ and Z² are each non-metallic atoms necessary for forming a substituted or unsubstituted condensed benzo ring or condensed naptho ring;

r is 0, 1 or 2;

L¹ to L⁷ are the same or different and each is a substituted or unsubstituted methine, provided that when r is 2, L⁶ and L⁷ that occur in duplicate are the same or different;

X and Y are the same or different and each is a group of the formula —O—, —S—, —CH═CH— or —C(R³R⁴)— wherein R³ and R⁴ are the same or different and each is substituted or unsubstituted alkyl.

WO 00/16810 teaches that r of formula A is preferably 1, i.e. the dyes are heptamethine cyanine dyes, and that preferred dyes having 3 or more sulfonic acid groups in the molecule are benzindole dyes of formula B:

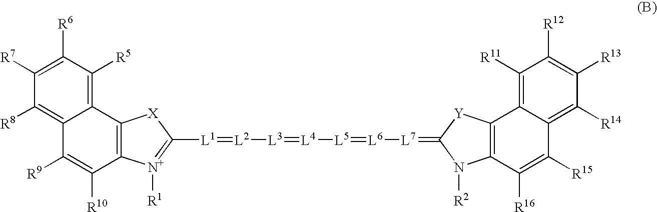

wherein R¹, R², L¹-L⁷, X and Y are as defined for formula A, and
R⁵ to R¹⁶ are the same or different and each is H, a sulfonic acid group, a carboxyl group, OH, an alkyl(sulfoalkyl)amino group, a bis(sulfoalkyl)amino group, a sulfoalkoxy group a (sulfoalkyl)sulfonyl group or a (sulfoalkyl)aminosulfonyl group, exclusive of several specific compounds.

The L¹ to L⁷ polymethine chain of WO 00/16810 is preferably of formula C:

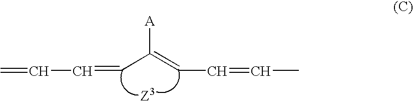

where Z³ is the non-metallic atoms necessary to form a 5- or 6-membered ring;
   A is H or a monovalent group.

WO 00/16810 teaches that, for superior water solubility the number of sulfonic acid groups is preferably 4 or more, but that for ease of synthesis the total number should be not more than 10, preferably no more than 8. WO 00/16810 also teaches preferred locations for the sulfonic acid groups:
formula A—positions R¹, R², Z¹ and/or Z².
formula B—positions R¹, R², R⁵, R⁷, R¹¹ and/or R¹³;
formula C—position A via a divalent group such as alkylene.

WO 01/43781 discloses cyanine dyes with 7 methine carbons (i.e. heptamethine or Cy7 dyes), corresponding to r=1 in formula A above. The dyes of WO 01/43781 have 4 to 6 sulfonic acid substituents.

Licha et al [Photochem.Photobiol., 72(3), 392-398 (2000)] report that cyanine dyes having at least one hydrophilic glucamide or glucosamide substituent exhibit reduced plasma protein binding (PPB) compared to the parent dye. Two such substituents instead of one is said to lower the PPB yet further. The hydrophilic substituents are also said to improve the photophysical properties of the dye, and alter the pharmacokinetics such that contrast between tumour and normal tissue is amplified.

U.S. Pat. No. 6,977,305 (Molecular Probes, Inc.) provides compounds of formula:

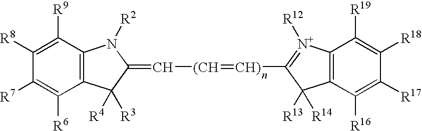

where:
R² and R¹² are independently alkyl or sulfoalkyl;
R³ is carboxyalkyl;
R⁴ R¹³ and R¹⁴ are independently alkyl;
R⁶ to R⁹ and R¹⁶ to R¹⁹ are independently H or sulfo; and
n is 1, 2 or 3.

Also disclosed are activated esters of the dyes. Related patent U.S. Pat. No. 6,974,873 discloses methods of staining biological samples using the dyes, as well as methods of forming dye-conjugates with proteins, peptides or a nucleic acid polymer using N-hydroxysuccinimide esters of the dyes.

WO 2005/044923 discloses dyes suitable for the labelling and detection of biological materials. The dyes are trimethine, pentamethine and heptamethine cyanine dyes (i.e. n is 1, 2 or 3) of formula D:

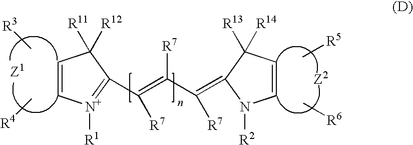

wherein:
R¹ and R² are C₁₋₆ alkyl; benzyl either unsubstituted or substituted with sulfonic aid or —(CH₂)ₖ—W;
   where W is a sulfonic acid or a phosphonic acid, and k is an integer of value 1 to 10;
R³ to R⁶ are H, SO₃H or -E-F;
   where E is a single bond or a spacer group having a chain of 1-20 linked atoms selected from C, N and O, and F is target bonding group;
R¹¹, R¹², R¹³ and R¹⁴ are C₁₋₆ alkyl or —(CH₂)ₖ—W;
Z¹ and Z² are independently the carbon atoms necessary to complete a one- or two-ring aromatic system;

with the provisos that:
(i) one or more of R¹¹, R¹², R¹³ and R¹⁴ is independently —(CH₂)ₖ—W,
(ii) at least one of R¹ to R⁷ is -E-F.

The target bonding group (F) of WO 2005/044923 is designed to react with a functional group of a target component (eg. a protein, peptide, nucleic acid or carbohydrate). WO 2005/044923 teaches that the presence of one or preferably multiple water-solubilising groups attached at the 3-position of the indolinium ring (ie. R¹¹ or R¹²) reduces dye-dye interactions, particularly when the dyes are attached to components such as nucleic acids, proteins, antibodies etc, and thus helps to minimise loss of fluorescence intensity due to dye-dye stacking. WO 2005/044923 teaches that W is preferably a sulfonic acid, and that at least 2 —(CH₂)ₖ—W groups should be present, which are preferably chosen such that one of the R¹¹/R¹² groups and one of the R¹³/R¹⁴ groups is —(CH₂)ₖ—W, and the other is preferably —CH₃. WO 2005/044923 teaches that W is preferably sulfonic acid, and k is preferably 3 or 4. In a further embodiment, WO 2005/044923 teaches that the dyes are preferably substituted with 3 to 5 sulfonic acid groups, and that the use of such dyes for labelling biological target molecules reduces loss of fluorescence due to dye-dye aggregation. WO 2005/044923 also discloses methods of labelling biological molecules with the dyes of formula D. WO 2005/044923 is directed towards in vitro dye applications, and is silent on in vivo applications.

WO 2005/123768 discloses conjugates of cyanine dyes (which are carbacyanines; oxacyanines, thiacyanines or azacyanines) with RGD type peptides for in vivo optical imaging of angiogenesis. The cyanine dyes of WO 2005/123768 are preferably pentamethine or heptamethine dyes, and preferably have zero, one or two sulfonic acid substituents. Reducing the number of sulfonate groups compared with prior art cyanine dyes is said to confer reduced plasma protein binding (PPB), and hence reduced non-specific uptake in vivo. Example 5 of WO 2005/123768 provides data on the PPB of the conjugates with pentamethine cyanine dyes having 1, 2 and 4 sulphonic acid groups. The PPB was found to increase with the number of sulphonic acid groups (PPB 17, 21 and 45% respectively).

Bullok et al [Biochem., 46(13), 4055-4065 (2007)] disclose an apoptosis probe TcapQ₅₄₇ which comprises an effector caspase recognition sequence (the tetrapeptide DEVD) conjugated to: (i) a membrane transporter peptide (Tat peptide); (ii) a far-red quencher (QSY 21) and (iii) the cyanine dye fluorophore Alexa Fluor™ 647. The intact probe exhibits very little fluorescence due to the quenching of QSY 21. After cleavage by caspases at sites of caspase activity, the cleaved peptide exhibits fluorescence due to the fact that the conjugated Alexa Fluor™ 647, is now in a different molecule to the quencher. The paper refers to studies both with separated, intact cells and an in vivo animal model.

Strong et al [Eur. Cytokine Netw., 17, 49-59 (2006)] disclose chemokine proteins modified with Alexa Fluor™ 647 at specific positions of their sequence. The specificity of cell staining in vitro was evaluated, leading the authors to suggest that the compounds could be useful in chemokine receptor assays based on intact cells.

### The Present Invention

The present invention provides imaging agents suitable for in vivo optical imaging, which comprise a specific class of pentamethine cyanine dye having a particular pattern of sulfonation, and conjugated to a biological targeting moiety (BTM). The present inventors have found that, for pentamethine dyes, sulfoalkyl groups have an important role in reducing plasma protein binding (PPB). This is important for both in vivo and in vitro applications, since it helps to suppress non-specific binding. It is hypothesised that this is due to the more 3-dimensional or ‘bulky’ nature of such modified dyes, as opposed to the essentially 2-dimensional (or ‘flat’) aryl sulfonated dyes (e.g. Cy5 and Cy5.5).

The present inventors have found that, even within a coherent series of pentamethine cyanine dyes, when conjugated to biological targeting molecules (eg. RGD peptides), there are significant variations in biological characteristics—in particular non-specific binding. This contributes to unwanted background uptake in vivo, and hence reduced image contrast plus slower background clearance requiring unwanted delay before imaging. In addition, and not recognised in the prior art, non-specific binding to collagen (which is widely distributed in the mammalian body), varies significantly.

The present invention provides a specific subset of pentamethine cyanine dyes which have preferred characteristics for in vivo imaging.

### DETAILED DESCRIPTION OF THE INVENTION

In a first aspect, the present invention provides an imaging agent suitable for in vivo optical imaging of the mammalian body which comprises a conjugate of Formula I:

[BTM]-(L)ₙ-Cy^{D} (I)
where:
BTM is a biological targeting molecule;
Cy^{D} is a cyanine dye of Formula II:

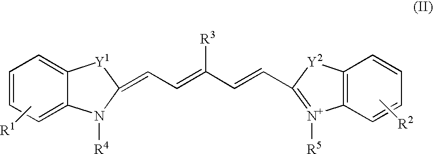

where:
   Y¹ and Y² are independently —O—, —S—, —NR⁶— or —CR⁷R⁸— and are chosen such that at least one of Y¹ and Y² is —CR⁷R⁸—;
   R¹ and R² are independently H, —SO₃M¹ or R^{a}, where M¹ is H or B^{c}, and B^{c} is a biocompatible cation;
   R³ is H, C₁₋₅ alkyl, C₁₋₆ carboxyalkyl or an R^{a} group;
   R⁴ to R⁶ are independently C₁₋₅ alkyl, C₁₋₆ carboxyalkyl or R^{a};
   R⁷ is C₁₋₃ alkyl;
   R⁸ is R^{a} or C₁₋₆ carboxyalkyl;
   R^{a} is C₁₋₄ sulfoalkyl;
   L is a synthetic linker group of formula -(A)ₘ- wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
   each R is independently chosen from H, C₁₋₄ alkyl, C₂₋₄ alkenyl, C₂₋₄ alkynyl, C₁₋₄ alkoxyalkyl or C₁₋₄ hydroxyalkyl;
   m is an integer of value 1 to 20;
   n is an integer of value 0 or 1;
with the provisos that:
(i) the cyanine dye comprises at least one R^{a} group and a total of 3 to 6 sulfonic acid substituents from the R¹, R² and R^{a} groups;
(ii) the imaging agent does not comprise a fluorescence quencher.

By the term “imaging agent” is meant a compound suitable for optical imaging of a region of interest of the whole (ie. intact) mammalian body in vivo. Preferably, the mammal is a human subject. The imaging may be invasive (eg. intra-operative or endoscopic) or non-invasive. The imaging may optionally be used to facilitate biopsy (eg. via a biopsy channel in an endoscope instrument), or tumour resection (eg. during intra-operative procedures via tumour margin identification).

Whilst the conjugate of Formula I is suitable for in vivo imaging, it may also have in vitro applications (eg. assays quantifying the BTM in biological samples or visualisation of BTM in tissue samples). Preferably, the imaging agent is used for in vivo imaging.

By the term “sulfonic acid substituent” is meant a substituent of formula —SO₃M¹, where M¹ is H or B^{c}, and B^{c} is a biocompatible cation. The —SO₃M¹, substituent is covalently bonded to a carbon atom, and the carbon atom may be aryl (such as the R¹ or R² groups), or alkyl (ie. an R^{a} group). By the term “biocompatible cation” (B^{c}) is meant a positively charged counterion which forms a salt with an ionised, negatively charged group (in this case a sulfonate group), where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

By the term “fluorescence quencher” is meant a moiety which suppresses the fluorescence of the Cy^{D} such that the BTM having both quencher and Cy^{D} attached would have minimal fluorescence. Quencher molecules are known in the art [Johansson, Meth. Mol. Biol., 335, 17-29 (2006), and Bullok et al (above)]. The imaging agent conjugates of the present invention are thus suitably already fluorescent due to the presence of the Cy^{D}, and do not need metabolic activation to separate the Cy^{D} from a quencher. This has the advantage that the BTM does not have conjugated thereto an additional molecule which might affect the capability of the BTM to interact with its biological recognition site in vivo—due to eg. steric hindrance or change in conformation due to the interaction between the quencher and the Cy^{D} or the quencher and the BTM or the quencher and the linker group. In addition, the need for a quencher limits the BTM to one that is a substrate for the biological target (ie. is cleaved enzymatically), or that undergoes a significant conformational change upon binding. Not having a quencher allows a greater range number of BTM to used, which in turn permits a greater range of disease states to be diagnosed. Any potential toxicity issues due to the quencher are also removed from consideration.

By the term “biological targeting moiety” (BTM) is meant a compound which, after administration, is taken up selectively or localises at a particular site of the mammalian body. Such sites may for example be implicated in a particular disease state be indicative of how an organ or metabolic process is functioning. The biological targeting moiety preferably comprises: 3-100 mer peptides, peptide analogue, peptoids or peptide mimetics which may be linear peptides or cyclic peptides or combinations thereof; or enzyme substrates, enzyme antagonists or enzyme inhibitors; synthetic receptor-binding compounds; oligonucleotides, or oligo-DNA or oligo-RNA fragments.

By the term “peptide” is meant a compound comprising two or more amino acids, as defined below, linked by a peptide bond (ie. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. The term “peptide analogue” refers to peptides comprising one or more amino acid analogues, as described below. See also “Synthesis of Peptides and Peptidomimetics”, M. Goodman et al, Houben-Weyl E22c, Thieme.

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

Suitable enzyme substrates, antagonists or inhibitors include glucose and glucose analogues such as fluorodeoxyglucose; fatty acids, or elastase, Angiotensin II or metalloproteinase inhibitors. A preferred non-peptide Angiotensin II antagonist is Losartan. Suitable synthetic receptor-binding compounds include estradiol, estrogen, progestin, progesterone and other steroid hormones; ligands for the dopamine D-1 or D-2 receptor, or dopamine transporter such as tropanes; and ligands for the serotonin receptor.

The cyanine dye (Cy^{D}) of Formula II is a fluorescent dye or chromophore which is capable of detection either directly or indirectly in an optical imaging procedure using light of green to near-infrared wavelength (500-1200 nm, preferably 600-1000 nm). Preferably, the Cy^{D} has fluorescent properties.

It is envisaged that one of the roles of the linker group -(A)ₘ- of Formula I is to distance the Cy^{D} from the active site of the BTM. This is particularly important because the Cy^{D} is relatively bulky, so adverse steric interactions are possible. This can be achieved by a combination of flexibility (eg. simple alkyl chains), so that the Cy^{D} has the freedom to position itself away from the active site and/or rigidity such as a cycloalkyl or aryl spacer which orientate the Cy^{D} away from the active site. The nature of the linker group can also be used to modify the biodistribution of the imaging agent. Thus, eg. the introduction of ether groups in the linker will help to minimise plasma protein binding. When -(A)ₘ- comprises a polyethyleneglycol (PEG) building block or a peptide chain of 1 to 10 amino acid residues, the linker group may function to modify the pharmacokinetics and blood clearance rates of the imaging agent in vivo. Such “biomodifier” linker groups may accelerate the clearance of the imaging agent from background tissue, such as muscle or liver, and/or from the blood, thus giving a better diagnostic image due to less background interference. A biomodifier linker group may also be used to favour a particular route of excretion, eg. via the kidneys as opposed to via the liver.

By the term “sugar” is meant a mono-, di- or tri-saccharide. Suitable sugars include: glucose, galactose, maltose, mannose, and lactose. Optionally, the sugar may be functionalised to permit facile coupling to amino acids. Thus, eg. a glucosamine derivative of an amino acid can be conjugated to other amino acids via peptide bonds. The glucosamine derivative of asparagine (commercially available from NovaBiochem) is one example of this:

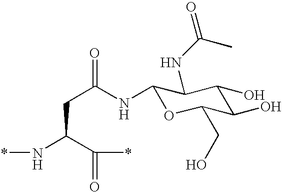

Formula I denotes that the -(L)ₙ[Cy^{D}] moiety can be attached at any suitable position of the BTM. Suitable such positions for the -(L)ₙ[Cy^{D}] moiety are chosen to be at positions away from that part of the BTM which is responsible for binding to the active site in vivo. The [BTM]-(L)ₙ- moiety of Formula I may be attached at any suitable position of the Cy^{D} of Formula II. The [BTM]-(L)ₙ- moiety either takes the place of an existing substituent (eg. one of the R¹ to R⁸ groups), or is covalently attached to the existing substituent of the Cy^{D}. The [BTM]-(L)ₙ- moiety is preferably attached via a carboxyalkyl substituent of the Cy^{D}.

Preferred Features.

The molecular weight of the imaging agent is suitably up to 30,000 Daltons. Preferably, the molecular weight is in the range 1,000 to 20,000 Daltons, most preferably 2000 to 18,000 Daltons, with 2,500 to 16,000 Daltons being especially preferred.

The BTM may be of synthetic or natural origin, but is preferably synthetic. The term “synthetic” has its conventional meaning, ie. man-made as opposed to being isolated from natural sources eg. from the mammalian body. Such compounds have the advantage that their manufacture and impurity profile can be fully controlled. Monoclonal antibodies and fragments thereof of natural origin are therefore outside the scope of the term ‘synthetic’ as used herein.

The BTM is preferably chosen from: a 3-100 mer peptide, enzyme substrate, enzyme antagonist or enzyme inhibitor. BTM is most preferably a 3-100 mer peptide or peptide analogue. When the BTM is a peptide, it is preferably a 4-30 mer peptide, and most preferably a 5 to 28-mer peptide.

In Formula II, Y¹ and Y² are preferably both independently —CR⁷R⁸—. In Formula II, R³ is preferably H or an R^{a} group, and is most preferably H. R⁷ is preferably CH₃.

The [BTM]-(L)ₙ- moiety of Formula I is preferably attached at positions R³, R⁴, R⁵, R⁶, R⁷ or R⁸ of the Cy^{D} of Formula II, more preferably at R³, R⁴ or R⁵, most preferably at R⁴ or R⁵. Attachment of the BTM at the R³ position has the advantages that:
(i) additional preferred sites for location of sulfoalkyl groups (R^{a}) are made available;
(ii) the bulkiness of the dye is increased, hence helping to reduce PPB.

The cyanine dye (Cy^{D}) preferably has a total of 4 sulfonic acid substituents chosen from the R¹, R² and R^{a} groups. The two R^{a} groups are preferably located at positions Y², R³, R⁴ or R⁵, most preferably at R⁵ together with either Y²═—CR⁷R^{a}— or R⁴═R^{a}. In Formula II, the R^{a} groups are preferably of formula —(CH₂)ₖSO₃M¹, where M¹ is H or B^{c}, k is an integer of value 1 to 4, and B^{c} is a biocompatible cation (as defined above). k is preferably 3 or 4.

In Formula II, R¹ and R² are preferably both SO₃M¹. When R¹ and R² are both SO₃M¹, the SO₃M¹ substituents are preferably in the 5-position of the indole/indolenine rings.

Especially preferred dyes are of Formula III:

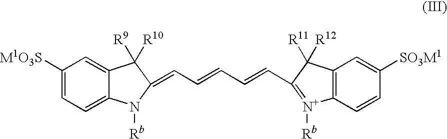

where:
R^{b} is independently an R^{a} group or C₁₋₆ carboxyalkyl;
R⁹ to R¹² are independently C₁₋₅ alkyl or an R^{b} group, and are chosen such that either R⁹═R¹⁰═R^{c} or R¹¹═R¹²═R^{c}, where R^{c} is C₁₋₂ alkyl;
R^{a} and M¹ are as defined above for Formula II.

The R^{a} groups of Formula III are preferably independently —(CH₂)ₖSO₃M¹, where k is an integer of value 1 to 4, and k is preferably 3 or 4. Preferably the dyes of Formula III have a C₁₋₆ carboxyalkyl substituent to permit facile covalent attachment to the BTM.

Preferred dyes of Formula III are chosen such that one of R⁹ to R¹² is an R^{b} group, and the others are each R^{c} groups, most preferably each equal to CH₃. Especially preferred dyes of Formula III are of Formula IIIa, wherein one of R⁹ to R¹² is an R^{a} group, and the others are each R^{c} groups, most preferably each equal to CH₃. Preferred dyes of Formula IIIa have one of the R^{b} groups chosen to be C₁₋₆ carboxyalkyl.

Most preferred specific dyes of Formulae III and IIIa respectively are Alexa Fluor™ 647 and Cy5**, with Cy5** being the ideal:

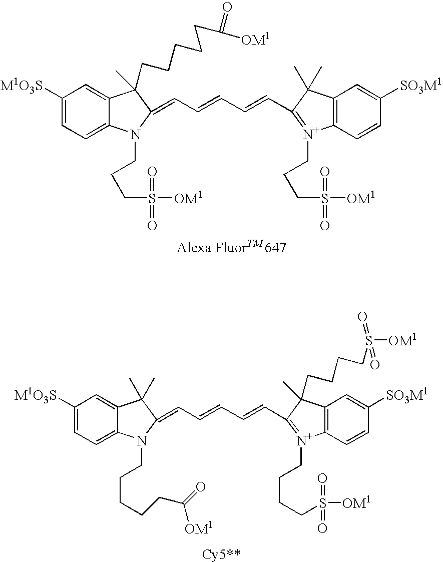

When the BTM is a peptide, preferred such peptides include:
somatostatin, octreotide and analogues,
peptides which bind to the ST receptor, where ST refers to the heat-stable toxin produced by E. coli and other micro-organisms;
laminin fragments eg. YIGSR, PDSGR, IKVAV, LRE and KCQAGTFALRGDPQG,
N-formyl peptides for targeting sites of leucocyte accumulation,
Platelet factor 4 (PF4) and fragments thereof,
RGD (Arg-Gly-Asp)-containing peptides, which may eg. target angiogenesis [R. Pasqualini et al., Nat Biotechnol. 1997 June; 15(6):542-6]; [E. Ruoslahti, Kidney Int. 1997 May; 51(5):1413-7].
peptide fragments of α₂-antiplasmin, fibronectin or beta-casein, fibrinogen or thrombospondin. The amino acid sequences of α₂-antiplasmin, fibronectin, beta-casein, fibrinogen and thrombospondin can be found in the following references: α₂-antiplasmin precursor [M. Tone et al., J. Biochem, 102, 1033, (1987)]; beta-casein [L. Hansson et al, Gene, 139, 193, (1994)]; fibronectin [A. Gutman et al, FEBS Lett., 207, 145, (1996)]; thrombospondin-1 precursor [V. Dixit et al, Proc. Natl. Acad. Sci., USA, 83, 5449, (1986)]; R. F. Doolittle, Ann. Rev. Biochem., 53, 195, (1984);
peptides which are substrates or inhibitors of angiotensin, such as: angiotensin II Asp-Arg-Val-Tyr-Ile-His-Pro-Phe (E. C. Jorgensen et al, J. Med. Chem., 1979, Vol 22, 9, 1038-1044) [Sar, Ile] Angiotensin II: Sar-Arg-Val-Tyr-Ile-His-Pro-Ile (R. K. Turker et al., Science, 1972, 177, 1203).
Angiotensin I: Asp-Arg-Val-Tyr-Ile-His-Pro-Phe-His-Leu;

When the BTM is a peptide, one or both termini of the peptide, preferably both, have conjugated thereto a metabolism inhibiting group (M^{IG}). Having both peptide termini protected in this way is important for in vivo imaging applications, since otherwise rapid metabolism would be expected with consequent loss of selective binding affinity for the BTM peptide. By the term “metabolism inhibiting group” (M^{IG}) is meant a biocompatible group which inhibits or suppresses enzyme, especially peptidase such as carboxypeptidase, metabolism of the BTM peptide at either the amino terminus or carboxy terminus. Such groups are particularly important for in vivo applications, and are well known to those skilled in the art and are suitably chosen from, for the peptide amine terminus:

N-acylated groups —NH(C═O)R^{G} where the acyl group —(C═O)R^{G} has R^{G} chosen from: C₁₋₆ alkyl, C₃₋₁₀ aryl groups or comprises a polyethyleneglycol (PEG) building block. Suitable PEG groups are described for the linker group (L), below. Preferred such PEG groups are the biomodifiers of Formulae Bio1 or Bio2 (below). Preferred such amino terminus M^{IG} groups are acetyl, benzyloxycarbonyl or trifluoroacetyl, most preferably acetyl.

Suitable metabolism inhibiting groups for the peptide carboxyl terminus include: carboxamide, tert-butyl ester, benzyl ester, cyclohexyl ester, amino alcohol or a polyethyleneglycol (PEG) building block. A suitable M^{IG} group for the carboxy terminal amino acid residue of the BTM peptide is where the terminal amine of the amino acid residue is N-alkylated with a C₁₋₄ alkyl group, preferably a methyl group. Preferred such M^{IG} groups are carboxamide or PEG, most preferred such groups are carboxamide.

When either or both peptide termini are protected with an M^{IG} group, the -(L)ₙ[Cy^{D}] moiety may optionally be attached to the M^{IG} group. Preferably, at least one peptide terminus has no M^{IG} group, so that attachment of the -(L)ₙ[Cy^{D}] moiety at that position gives compounds of Formulae IVa or IVb respectively:

[Cy^{D}]-(L)ₙ-[BTM]-Z² (IVa);

Z¹-{BTM]-(L)ₙ-[Cy^{D}] (IVb);

where:
Z¹ is attached to the N-terminus of the BTM peptide, and is H or M^{IG};
Z² is attached to the C-terminus of the BTM peptide and is OH, OB^{c}, or M^{IG}, where B^{c} is a biocompatible cation (as defined above).

In Formula IVa and IVb, Z¹ and Z² are preferably both independently M^{IG}. Preferred such M^{IG} groups for Z¹ and Z² are as described above for the peptide termini. Whilst inhibition of metabolism of the BTM peptide at either peptide terminus may also be achieved by attachment of the -(L)ₙ[Cy^{D}] moiety in this way, -(L)ₙ[Cy^{D}] itself is outside the definition of M^{IG} of the present invention.

The BTM peptide may optionally comprise at least one additional amino acid residue which possesses a side chain suitable for facile conjugation of the Cy^{D}, and forms part of the A residues of the linker group (L). Suitable such amino acid residues include Asp or Glu residues for conjugation with amine-functionalised Cy^{D} dyes, or a Lys residue for conjugation with a carboxy- or active ester-functionalised Cy^{D} dye. The additional amino acid residue(s) for conjugation of Cy^{D} are suitably located away from the binding region of the BTM peptide, and are preferably located at either the C- or N-terminus. Preferably, the amino acid residue for conjugation is a Lys residue.

When a synthetic linker group (L) is present, it preferably comprises terminal functional groups which facilitate conjugation to [BTM] and Cy^{D}. Suitable such groups (Q^{a}) are described in the fifth aspect (below). When L comprises a peptide chain of 1 to 10 amino acid residues, the amino acid residues are preferably chosen from glycine, lysine, arginine, aspartic acid, glutamic acid or serine. When L comprises a PEG moiety, it preferably comprises units derived from oligomerisation of the monodisperse PEG-like structures of Formulae Bio1 or Bio2:

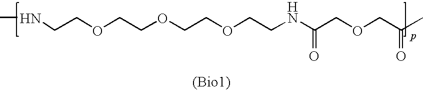

17-amino-5-oxo-6-aza-3,9,12,15-tetraoxaheptadecanoic acid of Formula Bio1 wherein p is an integer from 1 to 10. Alternatively, a PEG-like structure based on a propionic acid derivative of Formula Bio2 can be used:

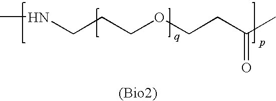

where p is as defined for Formula Bio 1 and q is an integer from 3 to 15.

In Formula Bio2, p is preferably 1 or 2, and q is preferably 5 to 12.

When the linker group does not comprise PEG or a peptide chain, preferred L groups have a backbone chain of linked atoms which make up the -(A)ₘ- moiety of 2 to 10 atoms, most preferably 2 to 5 atoms, with 2 or 3 atoms being especially preferred. A to minimum linker group backbone chain of 2 atoms confers the advantage that the Cy^{D} is well-separated so that any undesirable interaction is minimised.

BTM peptides which are not commercially available can be synthesised by solid phase peptide synthesis as described in P. Lloyd-Williams, F. Albericio and E. Girald; Chemical Approaches to the Synthesis of Peptides and Proteins, CRC Press, 1997.

The imaging agents can be prepared as follows:

In order to facilitate conjugation of the Cy^{D} to the BTM, the Cy^{D} suitably has attached thereto a reactive functional group (Q^{a}). The Q^{a} group is designed to react with a complementary functional group of the BTM, thus forming a covalent linkage between the Cy^{D} and the BTM. The complementary functional group of the BTM may be an intrinsic part of the BTM, or may be introduced by use of derivatisation with a bifunctional group as is known in the art. Table 1 shows examples of reactive groups and their complementary counterparts:

TABLE 1
Reactive Substituents and Complementary Groups Reactive Therewith.

| Reactive Group (Q^{a}) | Complementary Groups |
|---|---|
| Activated ester | primary amino, secondary amino |
| acid anhydride, acid halide. | primary amino, secondary amino, hydroxyl |
| isothiocyanate | amino groups |
| vinylsulphone | amino groups |
| dichlorotriazine | amino groups |
| haloacetamide, maleimide | thiol, imidazole, hydroxyl, amines, |
| | thiophosphate |
| carbodiimide | carboxylic acids |
| hydrazine, hydrazide | carbonyl including aldehyde and ketone |
| phosphoramidite | hydroxyl groups |

By the term “activated ester” or “active ester” is meant an ester derivative of the carboxylic acid which is designed to be a better leaving group, and hence permit more facile reaction with nucleophile, such as amines. Examples of suitable active esters are: N-hydroxysuccinimide (NHS), pentafluorophenol, pentafluorothiophenol, para-nitrophenol and hydroxybenzotriazole. Preferred active esters are N-hydroxysuccinimide or pentafluorophenol esters.

Examples of functional groups present in BTM such as proteins, peptides, nucleic acids carbohydrates and the like, include: hydroxy, amino, sulphydryl, carbonyl (including aldehyde and ketone) and thiophosphate. Suitable Q^{a} groups may be selected from: carboxyl; activated esters; isothiocyanate; maleimide; haloacetamide; hydrazide; vinylsulphone, dichlorotriazine and phosphoramidite. Preferably, Q^{a} is: an activated ester of a carboxylic acid, an isothiocyanate, a maleimide or a haloacetamide.

When the complementary group is an amine or hydroxyl, Q^{a} is preferably an activated ester, with preferred such esters as described above. A preferred such substituent on the Cy^{D} is the activated ester of a 5-carboxypentyl group. When the complementary group is a thiol, Q^{a} is preferably a maleimide or iodoacetamide group.

General methods for conjugation of cyanine dyes to biological molecules are described by Licha et al [Topics Curr. Chem., 222, 1-29 (2002); Adv. Drug Deliv. Rev., 57, 1087-1108 (2005)]. Peptide, protein and oligonucleotide substrates for use in the invention may be labelled at a terminal position, or alternatively at one or more internal positions. For reviews and examples of protein labelling using fluorescent dye labelling reagents, see “Non-Radioactive Labelling, a Practical Introduction”, Garman, A. J. Academic Press, 1997; “Bioconjugation—Protein Coupling Techniques for the Biomedical Sciences”, Aslam, M. and Dent, A., Macmillan Reference Ltd, (1998). Protocols are available to obtain site specific labelling in a synthesised peptide, for example, see Hermanson, G. T., “Bioconjugate Techniques”, Academic Press (1996).

Preferably, the method of preparation of the imaging agent comprises either:
(i) reaction of an amine functional group of a BTM with a compound of formula Y¹-(L)ₙ-[Cy^{D}]; or
(ii) reaction of a carboxylic acid or activated ester functional group of a BTM with a compound of formula Y²-(L)ₙ-[Cy^{D}];
(iii) reaction of a thiol group of a BTM with a compound of formula

Y³-(L)ₙ-[Cy^{D}];
wherein BTM, M^{IG}, L, n and Cy^{D} are as defined above, and
Y¹ is a carboxylic acid, activated ester, isothiocyanate or thiocyanate group;
Y² is an amine group;
Y³ is a maleimide group.

Y² is preferably a primary or secondary amine group, most preferably a primary amine group. In step (iii), the thiol group of the BTM is preferably from a cysteine residue.

In steps (i) to (iii), the BTM may optionally have other functional groups which could potentially react with the Cy^{D} derivative, protected with suitable protecting groups so that chemical reaction occurs selectively at the desired site only. By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Amine protecting groups are well known to those skilled in the art and are suitably chosen from: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl). Suitable thiol protecting groups are Trt (Trityl), Acm (acetamidomethyl), t-Bu (tert-butyl), tert-Butylthio, methoxybenzyl, methylbenzyl or Npys (3-nitro-2-pyridine sulfenyl). The use of further protecting groups are described in ‘Protective Groups in Organic Synthesis’, Theodora W. Greene and Peter G. M. Wuts, (John Wiley & Sons, 1991). Preferred amine protecting groups are Boc and Fmoc, most preferably Boc. Preferred amine protecting groups are Trt and Acm.

Cyanine dyes (Cy^{D}) functionalised suitable for conjugation to peptides are commercially available from GE Healthcare Limited, Atto-Tec, Dyomics, Molecular Probes and others. Most such dyes are available as NHS esters. Alexa Fluor™ 647 functionalised with hydrazide, maleimide or succinimidyl ester groups are commercially available from Molecular Probes. Cy^{D} functionalised at the R³ position with carboxyl or maleimide groups can be prepared in an analogous manner to that of EP 1816475 A1.

Methods of conjugating optical reporter dyes, to amino acids and peptides are described by Licha (vide supra), as well as Flanagan et al [Bioconj. Chem., 8, 751-756 (1997)]; Lin et al, [ibid, 13, 605-610 (2002)] and Zaheer [Mol. Imaging, 1(4), 354-364 (2002)]. Methods of conjugating the linker group (L) to the BTM employ analogous chemistry to that of the dyes alone (see above), and are known in the art.

Dyes of Formula III are described in the fifth aspect, below.

In a second aspect, the present invention provides a pharmaceutical composition which comprises the imaging agent of the first aspect together with a biocompatible carrier, in a form suitable for mammalian administration.

The “biocompatible carrier” is a fluid, especially a liquid, in which the imaging agent can be suspended or dissolved, such that the composition is physiologically tolerable, ie. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). Preferably the biocompatible carrier is pyrogen-free water for injection or isotonic saline.

The imaging agents and biocompatible carrier are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may optionally contain additional excipients such as an antimicrobial preservative, pH-adjusting agent, filler, stabiliser or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The pharmaceutical compositions of the second aspect may be prepared under aseptic manufacture (ie. clean room) conditions to give the desired sterile, non-pyrogenic product. It is preferred that the key components, especially the associated reagents plus those parts of the apparatus which come into contact with the imaging agent (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

The pharmaceutical composition of the second aspect is preferably prepared from a kit, as described for the third aspect below.

In a third aspect, the present invention provides a kit for the preparation of the pharmaceutical composition of the second aspect, which comprises the imaging agent of the first aspect in sterile, solid form such that, upon reconstitution with a sterile supply of the biocompatible carrier of the second aspect, dissolution occurs to give the desired pharmaceutical composition.

In that instance, the imaging agent, plus other optional excipients as described above, may be provided as a lyophilised powder in a suitable vial or container. The agent is then designed to be reconstituted with the desired biocompatible carrier to give the pharmaceutical composition in a sterile, apyrogenic form which is ready for mammalian administration.

A preferred sterile, solid form of the imaging agent is a lyophilised solid. The sterile, solid form is preferably supplied in a pharmaceutical grade container, as described for the pharmaceutical composition (above). When the kit is lyophilised, the formulation may optionally comprise a cryoprotectant chosen from a saccharide, preferably mannitol, maltose or tricine.

In a fourth aspect, the present invention provides a conjugate of Formula Ia:

[BTM]-(L)ₙ-Cy^{D} (Ia)
where: BTM, L and n are as defined for the first aspect, and Cy^{D} is of Formula IIIc:

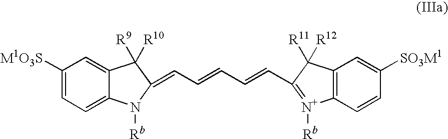

where:
R⁹ to R¹² are independently R^{b} or R^{c} groups, and are chosen such that
   one of R⁹═R¹⁰ is an R^{a} group, and the others are each R^{c} groups, where R^{c} is C₁₋₂ alkyl;
R^{a}, R^{b} and M¹ are as defined for Formula III.

Preferred embodiments of Formula Ma in the conjugate are as described above.

The conjugates of the fourth aspect are useful in the preparation of both imaging agents and pharmaceutical compositions having the preferred cyanine dyes of Formula IIIa. Preferred aspects of the BTM, L, n and dye of Formula IIIa are as described above. The conjugates can be prepared as described in the first and fifth aspects.

In a fifth aspect, the present invention provides a cyanine dye of Formula IIIa as defined in the fourth aspect. The dyes of the fifth aspect are useful in the preparation of BTM-conjugates, imaging agents and pharmaceutical compositions having the preferred cyanine dyes of Formula IIIa.

Preferred aspects of the cyanine dye of Formula IIIa are as described above. The dyes preferably further comprise a group Q^{a}, where Q^{a} is a reactive functional group suitable for conjugation to BTM. Suitable and preferred Q^{a} groups are as described above. Dyes of Formula IIIa can be prepared as described for Cy5** in Example 3. Such dyes incorporating Q^{a} groups, where Q^{a} is an active ester, can be prepared according to Example 4.

In a sixth aspect, the present invention provides a method of in vivo optical imaging of the mammalian body which comprises use of either the imaging agent of the first aspect or the pharmaceutical composition of the second aspect to obtain images of sites of BTM localisation in vivo.

By the term “optical imaging” is meant any method that forms an image for detection, staging or diagnosis of disease, follow up of disease development or for follow up of disease treatment based on interaction with light in the green to near-infrared region (wavelength 500-1200 nm). Optical imaging further includes all methods from direct visualization without use of any device and involving use of devices such as various scopes, catheters and optical imaging equipment, eg. computer-assisted hardware for tomographic presentations. The modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching. Further details of these techniques are provided by: (Tuan Vo-Dinh (editor): “Biomedical Photonics Handbook” (2003), CRC Press LCC; Mycek & Pogue (editors): “Handbook of Biomedical Fluorescence” (2003), Marcel Dekker, Inc.; Splinter & Hopper: “An Introduction to Biomedical Optics” (2007), CRC Press LCC.

The green to near-infrared region light is suitably of wavelength 500-1200 nm, preferably of wavelength 600-1000 nm. The optical imaging method is preferably fluorescence endoscopy. The mammalian body of the sixth aspect is preferably the human body. Preferred embodiments of the imaging agent are as described for the first aspect (above). In particular, it is preferred that the Cy^{D} dye employed is fluorescent.

In the method of the sixth aspect, the imaging agent or pharmaceutical composition has preferably been previously administered to said mammalian body. By “previously administered” is meant that the step involving the clinician, wherein the imaging agent is given to the patient eg. as an intravenous injection, has already been carried out prior to imaging. This embodiment includes the use of the imaging agent of the first embodiment for the manufacture of a diagnostic agent for the diagnostic imaging in vivo of disease states of the mammalian body where the BTM is implicated.

A preferred optical imaging method of the sixth aspect is Fluorescence Reflectance Imaging (FRI). In FRI, the imaging agent of the present invention is administered to a subject to be diagnosed, and subsequently a tissue surface of the subject is illuminated with an excitation light—usually continuous wave (CW) excitation. The light excites the Cy^{D} dye of the imaging agent. Fluorescence from the imaging agent, which is generated by the excitation light, is detected using a fluorescence detector. The returning light is preferably filtered to separate out the fluorescence component (solely or partially). An image is formed from the fluorescent light. Usually minimal processing is performed (no processor to compute optical parameters such as lifetime, quantum yield etc.) and the image maps the fluorescence intensity. The imaging agent is designed to concentrate in the disease area, producing higher fluorescence intensity. Thus the disease area produces positive contrast in a fluorescence intensity image. The image is preferably obtained using a CCD camera or chip, such that real-time imaging is possible.

The wavelength for excitation varies depending on the particular Cy^{D} dye used, but is typically in the range 500-1200 nm for dyes of the present invention. The apparatus for generating the excitation light may be a conventional excitation light source such as: a laser (e.g., ion laser, dye laser or semiconductor laser); halogen light source or xenon light source. Various optical filters may optionally be used to obtain the optimal excitation wavelength.

A preferred FRI method comprises the steps as follows:
(i) a tissue surface of interest within the mammalian body is illuminated with an excitation light;
(ii) fluorescence from the imaging agent, which is generated by excitation of the Cy^{D}, is detected using a fluorescence detector;
(iii) the light detected by the fluorescence detector is optionally filtered to separate out the fluorescence component;
(iv) an image of said tissue surface of interest is formed from the fluorescent light of steps (ii) or (iii).

In step (i), the excitation light is preferably continuous wave (CW) in nature. In step (iii), the light detected is preferably filtered. An especially preferred FRI method is fluorescence endoscopy.

An alternative imaging method of the sixth aspect uses FDPM (frequency-domain photon migration). This has advantages over continuous-wave (CW) methods where greater depth of detection of the dye within tissue is important [Sevick-Muraca et al, Curr. Opin. Chem. Biol., 6, 642-650 (2002)]. For such frequency/time domain imaging, it is advantageous if the Cy^{D} has fluorescent properties which can be modulated depending on the tissue depth of the lesion to be imaged, and the type of instrumentation employed.

The FDPM method is as follows:
(a) exposing light-scattering biological tissue of said mammalian body having a heterogeneous composition to light from a light source with a pre-determined time varying intensity to excite the imaging agent, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

The fluorescence characteristic of step (c) preferably corresponds to uptake of the imaging agent and preferably further comprises mapping a number of quantities corresponding to adsorption and scattering coefficients of the tissue before administration of the imaging agent. The fluorescence characteristic of step (c) preferably corresponds to at least one of fluorescence lifetime, fluorescence quantum efficiency, fluorescence yield and imaging agent uptake. The fluorescence characteristic is preferably independent of the intensity of the emission and independent of imaging agent concentration.

The quantifying of step (c) preferably comprises: (i) establishing an estimate of the values, (ii) determining a calculated emission as a function of the estimate, (iii) comparing the calculated emission to the emission of said detecting to determine an error, (iv) providing a modified estimate of the fluorescence characteristic as a function of the error. The quantifying preferably comprises determining the values from a mathematical relationship modelling multiple light-scattering behaviour of the tissue. The method of the first option preferably further comprises monitoring a metabolic property of the tissue in vivo by detecting variation of said fluorescence characteristic.

The optical imaging of the sixth aspect is preferably used to help facilitate the management of a disease state of the mammalian body. By the term “management” is meant use in the: detection, staging, diagnosis, monitoring of disease progression or the monitoring of treatment. The disease state is suitably one in which the BTM of the imaging agent is implicated. Imaging applications preferably include camera-based surface imaging, endoscopy and surgical guidance. Further details of suitable optical imaging methods have been reviewed by Sevick-Muraca et al [Curr. Opin. Chem. Biol., 6, 642-650 (2002)].

In a further aspect, the present invention provides a method of detection, staging, diagnosis, monitoring of disease progression or monitoring of treatment of a disease state of the mammalian body which comprises the in vivo optical imaging method of the sixth aspect.

The invention is illustrated by the non-limiting Examples detailed below. Examples 1a and 2 provide the syntheses of Compounds 1 and 3 respectively, which are comparative Examples of related dyes outside the scope of the present claims. Example 1b provides the synthesis of Compound 2, which is a dye conjugate of a control peptide (scrambled RGD). Example 3 provides the synthesis of cyanine dye Cy5**, a preferred Cy^{D} of the invention. Example 4 provides the synthesis of an active ester of Cy5**. Example 5 provides the synthesis of Compound 4, a peptide conjugate of Cy5**. Example 6 provides the synthesis of Compound 6, a peptide conjugate of Alexa647. Example 7 provides plasma stability data for Compounds 1 to 8. All conjugates exhibited satisfactory plasma stability except Compounds 5 and 7 (46 and 70% of main peak left after 4 h incubation in plasma, respectively). Example 8 provides PPB data for compounds of the invention. The highest PPB was observed for Compounds 3 and 7, and the lowest for Compounds 4 and 6. Example 9 provides collagen binding data for Compounds 1 to 8. Most of the compounds showed a high degree of binding at low concentrations, whereas Compounds 4 and 6 exhibited the lowest collagen binding. Example 10 provides binding assay data on Compounds 1 to 8. All exhibited similar Ki values in the sub-nM range, except for Compound 7, which shows a slightly higher Ki value, and for Compound 2 (a scrambled negative control). This shows that biological binding properties are retained for an RGD peptide, despite the conjugation of a cyanine dye, and that this holds true for a range of cyanine dyes. Example 11 provides in vivo imaging data for Compounds 1 to 8. The analysis software assumes a simple exponential washout of the dye. The estimated washout times were found to be inaccurate, particularly for the skin and muscle signal where they probably are underestimated. This is believed to be due to the RGD binding to integrins and possibly collagen in the background tissue, giving an apparent double exponential washout characteristics. Slower wash-in and washout in the tumour compared to the muscle was considered favourable. The negative control (Compound 2) showed similar kinetics in the tumour and reference tissues, indicating that the observed differences with the positive compounds are due to targeting and not perfusion effects. Compound 6 was considered to have the most favourable imaging kinetics.

TABLE 2
Compounds of the invention.

| Compound | Dye | Vector |
|---|---|---|
| 1 | Cy5(2) | RGD |
| 2 | Cy5(2) | neg-RGD |
| 3 | Cy5(1) | RGD |
| 4 | Cy5** | RGD |
| 5 | Cy5*B | RGD |
| 6 | Alexa647 | RGD |
| 7 | Cy5*F | RGD |
| 8 | Cy5-PEG | RGD |

where: the RGD peptide used is given in Example 1, neg-RGD is a scrambled RGD peptide described in Example 1b, the dye structures are given in Table 3.

TABLE 3
Structures of Cyanine dyes of the Examples.

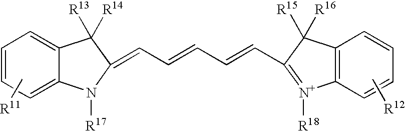

| | Dye name | | | | | | |
|---|---|---|---|---|---|---|---|
| | Cy5(1) | Cy5(2) | Cy5*B | Cy5*F | Cy5PEG | Cy5** | Alexa647 |
| R¹¹ | H | SO₃H | SO₃H | 4 × F | SO₃H | SO₃H | SO₃H |
| R¹² | SO₃H | SO₃H | SO₃H | 4 × F | SO₃H | SO₃H | SO₃H |
| R¹³ | CH₃ | CH₃ | CH₃ | CH₃ | CH₃ | CH₃ | R^{f} |
| R¹⁴ | CH₃ | CH₃ | R^{e} | R^{e} | CH₃ | CH₃ | CH₃ |
| R¹⁵ | CH₃ | CH₃ | CH₃ | CH₃ | CH₃ | CH₃ | CH₃ |
| R¹⁶ | CH₃ | CH₃ | R^{e} | R^{e} | CH₃ | R^{e} | CH₃ |
| R¹⁷ | R^{f} | R^{f} | R^{f} | R^{e} | Et | R^{f} | R^{d} |
| R¹⁸ | CH₃ | Et | benzyl | R^{f} | R^{p} | R^{e} | R^{d} |

where:
Cy5(1), Cy5(2), Cy5*B, Cy5*F and Cy5PEG are comparative examples
R^{d} is —(CH₂)₃SO₃H,
R^{e} is —(CH₂)₄SO₃H and
R^{f} is —(CH₂)₅CO₂H.
R^{p} is (CH₂)₅CONH(CH₂CH₂O)₃CH₂CH₂NHCOCH₂OCH₂CO₂H.

### ABBREVIATIONS

Conventional 3-letter and single letter amino acid abbreviations are used.

Acm: Acetamidomethyl

ACN: Acetonitrile

Boc: tert-Butyloxycarbonyl

DMF: N,N′-Dimethylformamide

DMSO: Dimethylsulfoxide

Fmoc: 9-Fluorenylmethoxycarbonyl

HCl: Hydrochloric acid

HPLC: High performance liquid chromatography

HSPyU O-(N-succinimidyl)-N,N,N′,N′-tetramethyleneuronium hexafluorophosphate

Ile: Isoleucine

LC-MS: Liquid chromatography mass spectroscopy

NHS: N-hydroxy-succinimide.

NMM: N-Methylmorpholine.

NMP: 1-Methyl-2-pyrrolidinone.

Pbf: 2,2,4,6,7-Pentamethyldihydrobenzofuran-5-sulfonyl.

PBS: Phosphate-buffered saline.

PPB: Plasma protein binding.

TFA: Trifluoroacetic acid.

Trt: Trityl.

TSTU: O-(N-Succinimidyl)-N,N,N′,N′-tetramethyluronium tetrafluoroborate.

### Example 1a

### Synthesis of RGD-[Cy5(2)] Dye Conjugate (Compound 1, Comparative Example)

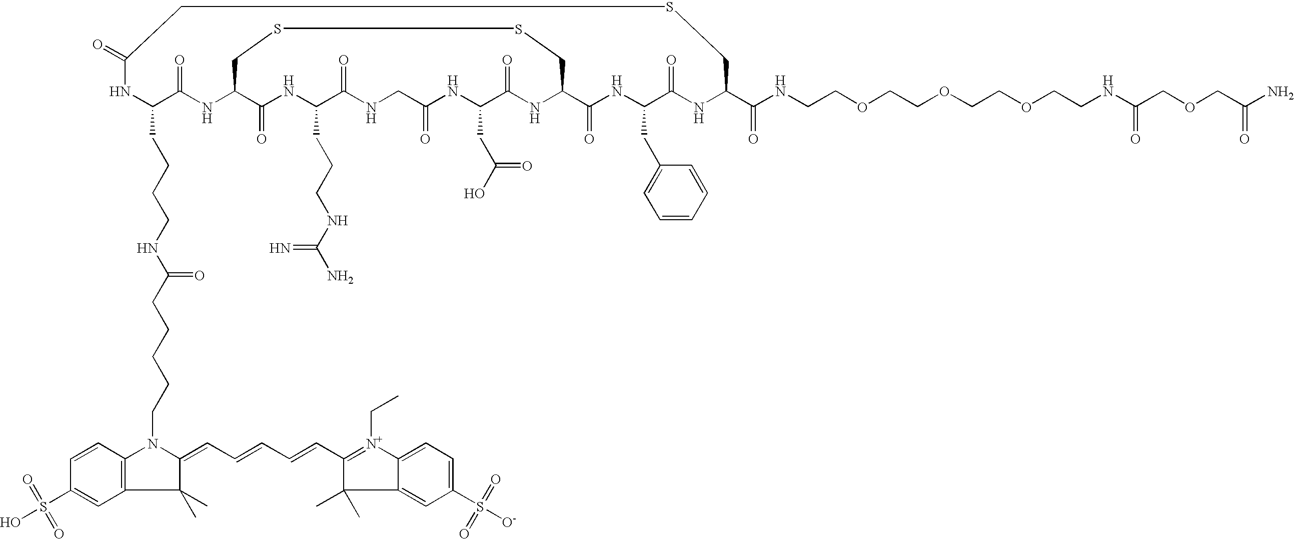

The RGD peptide (ref. WO 2005/123768; 24 mg, 0.02 mmol) was added as a solid to a solution of Cy5(2) mono NETS-ester (GE Healthcare Catalogue number PA15104; 7.5 mg, 0.01 mmol) in DMF (2 ml), and NMM (0.01 ml, 0.09 mmol) was then added. The reaction was allowed to proceed overnight with exclusion of light. The DMF was evaporated under reduced pressure and the crude product was purified by reverse phase preparative chromatography (Vydac C18 column, 218TP1022; solvents: A=water/0.1% TFA and B=CH₃CN/0.1% TFA; gradient 10-30% B over 60 min; flow 10 ml/min; detection at 254 nm), affording 6.6 mg (37%) of pure product (analytical HPLC: Phenomenex Luna C18 column, 00G-4252-E0; solvents: A=water/0.1% TFA and B=CH₃CN/0.1% TFA; gradient 15-35% B over 20 min; flow 1.0 ml/min; retention time 19.5 min; detection at 214 and 254 nm). Further characterisation was carried out using mass spectrometry, giving m/z value 949.1 [MH²⁺].

### Example 1b

### Synthesis of neg-RGD-[Cy5(2)] Dye Conjugate (Compound 2, Comparative Example)

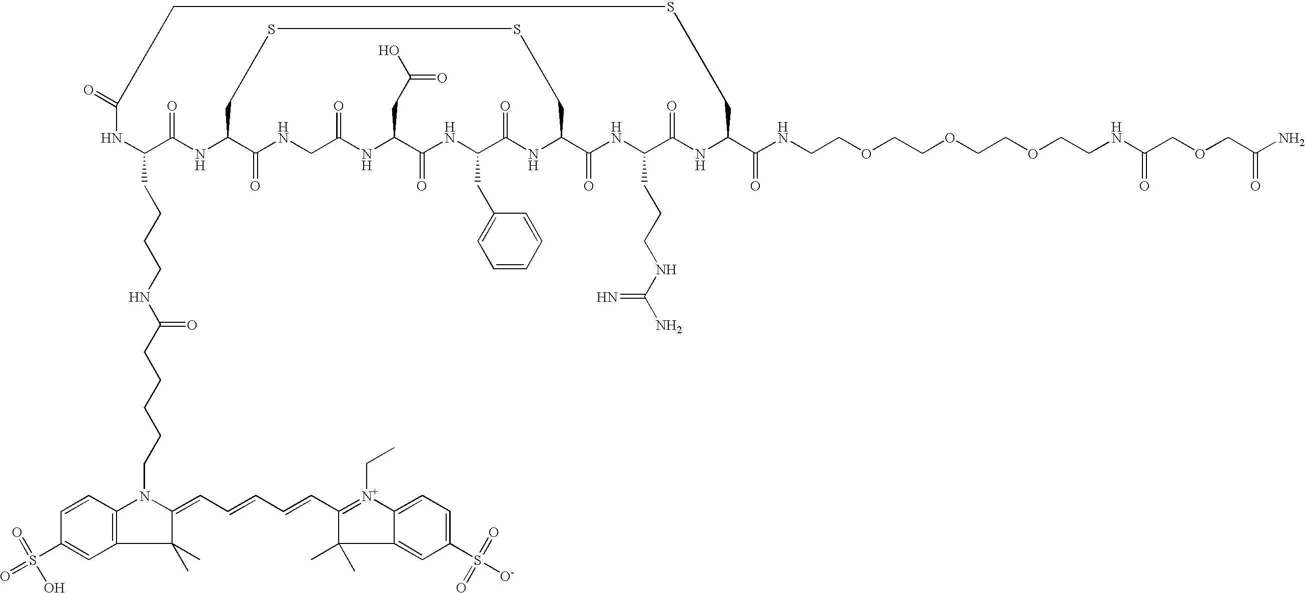

The neg-RGD peptide, containing the peptide sequence Lys-Cys-Gly-Asp-Phe-Cys-Arg-Cys, was prepared as described for the RDG peptide (ref. WO 2005/123768). Neg-RGD-[Cy5(2)] dye conjugate was prepared as described in Example 1. Crude product was purified by reverse phase preparative chromatography (Phenomenex Luna 5μ C18 (2) 250×21.20 mm; solvents: A=water/0.1% TFA and B=CH₃CN/0.1% TFA; gradient 20-30% B over 40 min; flow 10 ml/min; detection at 214 nm), affording 4.1 mg of title compound (analytical HPLC: Phenomenex Luna 3□ C18 (2) 20×2 mm; solvents: A=water/0.1% TFA and B=CH₃CN/0.1% TFA; gradient 10-40% B over 20 min; flow 1.0 ml/min; retention time 3.23 min; detection at 214 and 254 nm). Further characterisation was carried out using mass spectrometry, giving m/z value 1895.6 [M⁺].

### Example 2

### Synthesis of RGD-Cy5(1)) Dye Conjugate (Compound 3, Comparative Example)

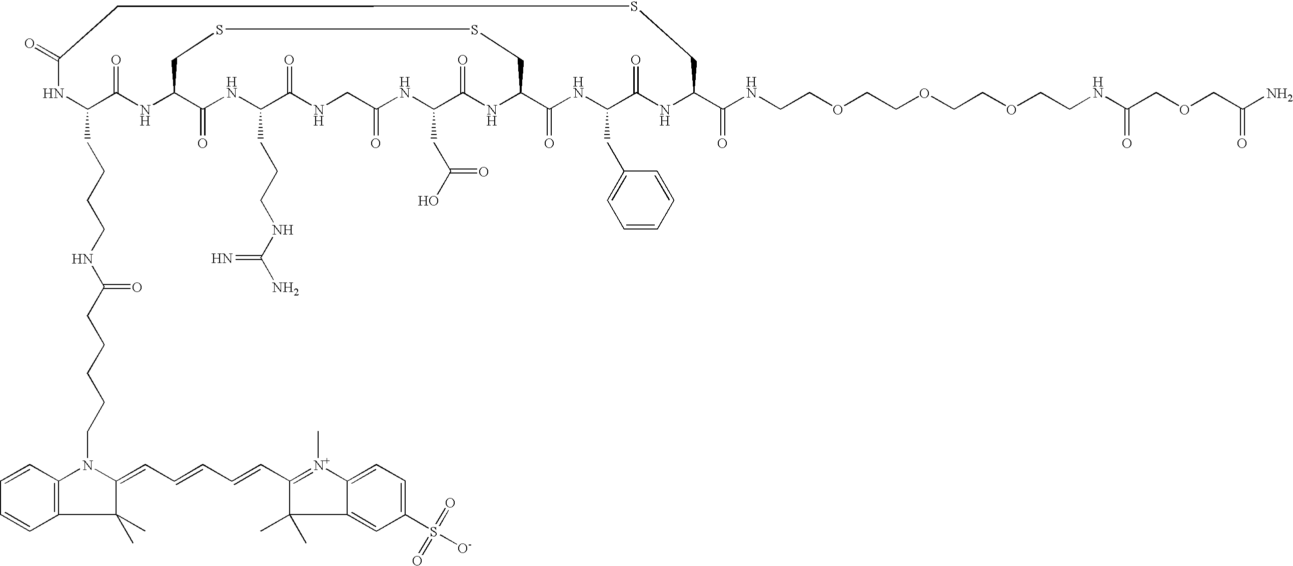

The NHS-ester of Cy5(1) (4.5 mg, 0.008 mmol) was formed by treatment of Cy5(1) with TSTU (2.1 mg, 0.0076 mmol) and NMM (0.009 ml, 0.08 mmol) in DMF (2 ml) for 1 h. The solution was then added to the RGD peptide (Example 1; 20 mg, 0.016 mmol) and the reaction was allowed to proceed overnight with exclusion of light. The DMF was evaporated under reduced pressure and the crude product was purified by reverse phase preparative chromatography (Vydac C18 column, 218TP1022; solvents: A=water/0.1% TFA and B=CH₃CN/0.1% TFA; gradient 20-40% B over 60 min; flow 10 ml/min; detection at 254 nm), affording 4.9 mg (34%) of pure product (analytical HPLC: Phenomenex Luna C18 column, 00G-4252-E0; solvents: A=water/0.1% TFA and B=CH₃CN/0.1% TFA; gradient 25-45% B over 20 min; flow 1.0 ml/min; retention time 15.2 min; detection at 214 and 254 nm). Further characterisation was carried out using mass spectrometry, giving m/z value 902.1 [MH²⁺].

### Example 3

### Synthesis of the Cyanine Dye 2-{(1E,3E,5E)-5-[1-(5-carboxypentyl)-3,3-dimethyl-5-sulfo-1,3-dihydro-2H-indol-2-ylidene]penta-1,3-dienyl}-3-methyl-1,3-bis(4-sulfobutyl)-3H-indolium-5-sulfonate (Cy5**)

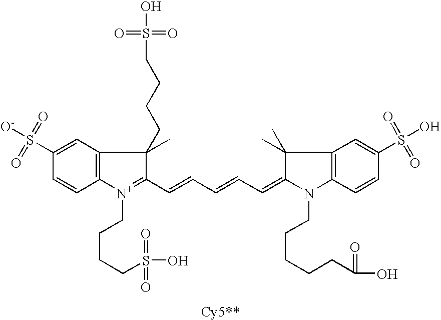

### (3a) 5-Methyl-6-oxoheptane-1-sulfonic acid

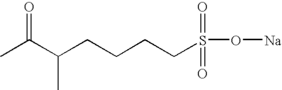

Ethyl 2-methylacetoacetate (50 g) in DMF (25 ml) was added to a suspension of sodium hydride (12.0 g of 60% NaH in mineral oil) in DMF (100 ml), dropwise with ice-bath cooling over 1 hour, (internal temperature 0-4° C.). This mixture was allowed to warm to ambient temperature for 45 mins with stirring before re-cooling. A solution of 1,4-butanesultone (45 g) in DMF (25 ml) was then added dropwise over 15 minutes. The final mixture was heated at 60° C. for 18 hours. The solvent was removed by rotary evaporation and the residue partitioned between water and diethyl ether. The aqueous layer was collected, washed with fresh diethyl ether and rotary evaporated to yield a sticky foam. This intermediate was dissolved in water (100 ml) and sodium hydroxide (17.8 g) added over 15 minutes with stirring. The mixture was heated at 90° C. for 18 hours. The cooled reaction mixture was adjusted to ˜pH2 by the addition of concentrated hydrochloric acid (˜40 ml). The solution was rotary evaporated and dried under vacuum. The yellow solid was washed with ethanol containing 2% hydrochloric acid (3×150 ml). The ethanolic solution was filtered, rotary evaporated and dried under vacuum to yield a yellow solid. Yield 70 g.

### (3b) 2,3-Dimethyl-3-(4-sulfobutyl)-3H-indole-5-sulfonic acid, dipotassium salt

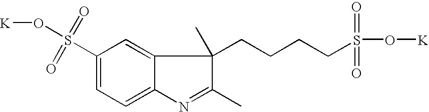

4-Hydrazinobenzenesulfonic acid (40 g), 5-methyl-6-oxoheptane-1-sulfonic acid (from 3a; 60 g) and acetic acid (500 ml) were mixed and heated under reflux for 6 hrs. The solvent was filtered, rotary evaporated and dried under vacuum. The solid was dissolved in methanol (1L). To this was added 2M methanolic potassium hydroxide (300 ml). The mixture was stirred for 3 hours and then the volume of solvent reduced by 50% using rotary evaporation. The resulting precipitate was filtered, washed with methanol and dried under vacuum. Yield 60 g. MS (LCMS): MH⁺ 362. Acc. Mass: Found, 362.0729. MH⁺═C₁₄H₂₀NO₆S₂ requires m/z 362.0732 (−0.8 ppm).

### (3c) 2,3-Dimethyl-1,3-bis(4-sulfobutyl)-3H-indolium-5-sulfonate, dipotassium salt

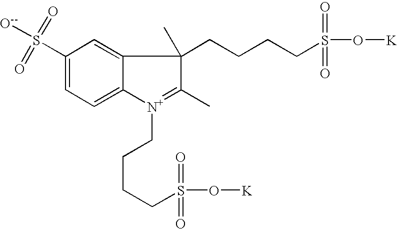

2,3-Dimethyl-3-(4-sulfobutyl)-3H-indole-5-sulfonic acid (from 3b; 60 g) was heated with 1,4 butane sultone (180 g) and tetramethylene sulfone (146 ml) at 140° C. for 16 hours. The resulting red solid was washed with diethyl ether, ground into a powder and dried under vacuum. Yield 60 g

### (3d) Cy5**, as TFA salt

1-(5′-Carboxypentyl)-2,3,3-trimethyl-indolenium bromide-5-sulfonic acid, K⁺ salt (2.7 g), malonaldehyde bis(phenylimine)monohydrochloride (960 mg), acetic anhydride (36 ml) and acetic acid (18 ml) were heated at 120° C. for 1 hour to give a dark brown-red solution. The reaction mixture was cooled to ambient temperature. 2,3-Dimethyl-1,3-bis(4-sulfobutyl)-3H-indolium-5-sulfonate (from 3c; 8.1 g) and potassium acetate (4.5 g) were added to the mixture, which was stirred for 18 hours at ambient temperature. The resulting blue solution was precipitated using ethyl acetate and dried under vacuum. The crude dye was purified by liquid chromatography (RPC₁₈. Water+0.1% TFA/MeCN+0.1% TFA gradient). Fractions containing the principal dye peak were collected, pooled and evaporated under vacuum to give the title dye, 2 g. UV/Vis (Water+0.1% TFA): 650 nm. MS (MALDI-TOF): MH+887.1. MH⁺═C₃₈H₅₀N₂O₁₄S₄ requires m/z 887.1

### Example 4

### Synthesis of 2-[(1E,3E,5E)-5-(1-{6-[(2,5-dioxopyrrolidin-1-yl)oxy]-6-oxohexyl}-3,3-dimethyl-5-sulfo-1,3-dihydro-2H-indol-2-ylidene)penta-1,3-dienyl]-3-methyl-1,3-bis(4-sulfobutyl)-3H-indolium-5-sulfonate, diisopropylethylamine salt (NHS Ester of Cy5**)

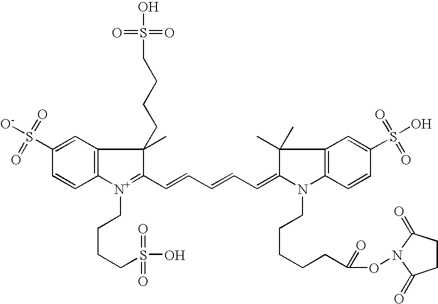

Cy5** (Example 3; 10 mg) was dissolved in anhydrous DMSO (3 ml); to this were added HSPyU (20 mg) and N,N′-diisopropylethylamine (80 μl). The resulting solution was mixed for 3 hours, whereupon TLC (RPC18. Water/MeCN) revealed complete reaction. The dye was isolated by precipitation in ethyl acetate/diethyl ether, filtered, washed with ethyl acetate and dried under vacuum. UV/Vis (Water) 650 nm. MS (MALDI-TOF) MH+983.5. MH⁺═C₄₂H₅₃N₃O₁₆S₄ requires m/z 984.16.

### Example 5

### Synthesis of the RGD-Cy5** Dye Conjugate (Compound 4)

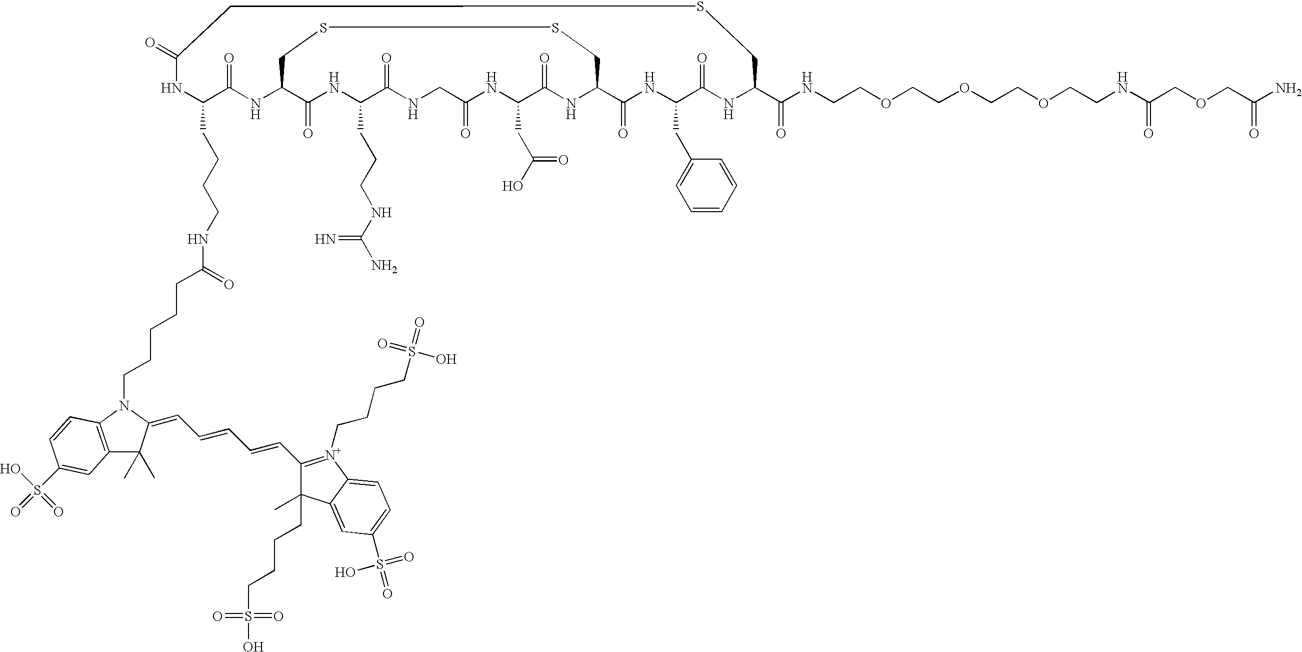

A solution of Cy5** NHS ester (2 mg, from Example 4) and sym-collidine (2 μL) dissolved in NMP (1 mL) was added dropwise to a solution of RGD peptide (from Example 1, 6.4 mg) and sym-collidine (2 μL) dissolved in DMF (1 mL) and the reaction mixture stirred overnight. The mixture was then diluted with 10% ACN/water/0.1% TFA (6 mL) and the product purified using preparative HPLC (Phenomenex Luna 5μ C18 (2) 250×21.20 mm column; solvents: A=water/0.1% TFA and B=CH₃CN/0.1% TFA; gradient 10-20% B over 40 min; flow 10 ml/min; detection at 214 nm), affording 2.3 mg (72%) of pure product (analytical HPLC: Phenomenex Luna 3μ C18 (2) 20×2 mm column; solvents: A=water/0.1 TFA and B=CH₃CN/0.1% TFA; gradient 10-40% B over 5 min; flow 0.6 ml/min; retention time 2.28 min; detection at 214 and 254 nm). Further characterisation was carried out using mass spectrometry, giving m/z value 1064.5 [MH²⁺].

### Example 6

### Synthesis of the RGD-Alexa 647 Dye Conjugate (Compound 6)

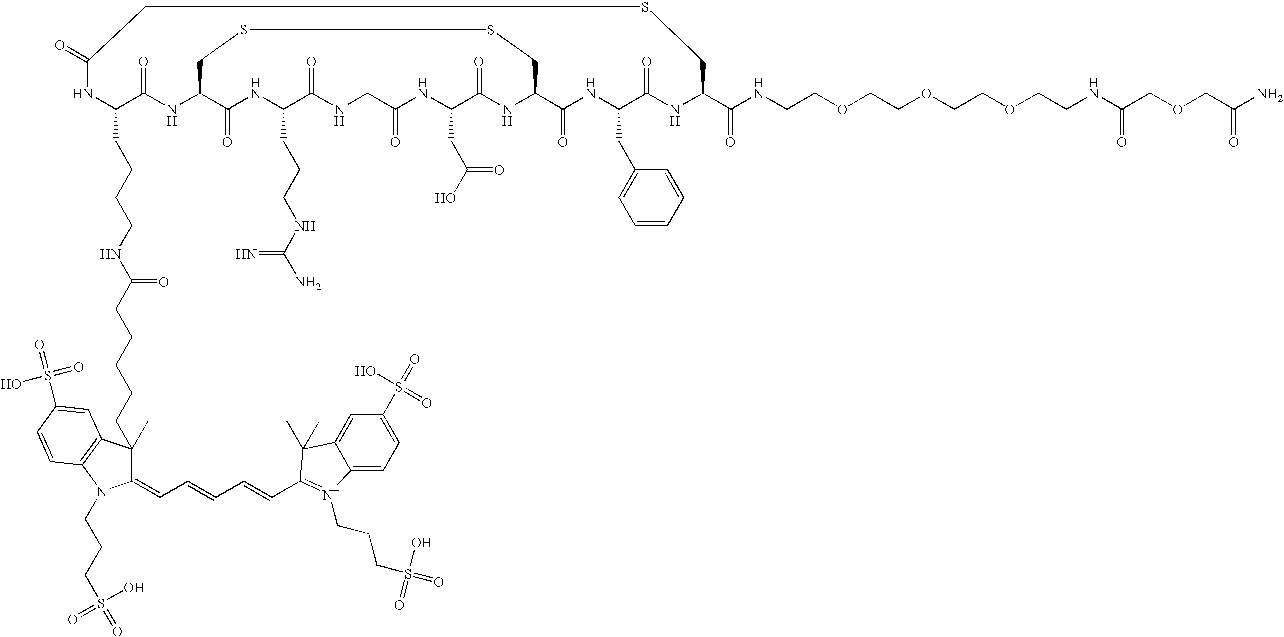

A solution of Alexa Fluor 647 NHS ester (2 mg, Molecular Probes A20106) and sym-collidine (3.2 μL) dissolved in NMP (1.4 mL) was added dropwise to a solution of RGD peptide (Example 1; 15 mg) and sym-collidine (3.2 μL) dissolved in DMF (1 mL) and the reaction mixture stirred overnight. The mixture was then diluted with water/0.1% TFA (6 mL) and the product purified using preparative HPLC (Phenomenex Luna 5μ C18 (2) 250×21.20 mm column; solvents: A=water/0.1% TFA and B=CH₃CN/0.1% TFA; gradient 10-25% B over 40 min; flow 10 ml/min; detection at 214 nm), affording 2.7 mg (53%) of pure product (analytical HPLC: Phenomenex Luna 3μ C18 (2) 20×2 mm column; solvents: A=water/0.1% TFA and B=CH₃CN/0.1% TFA; gradient 10-40% B over 5 min; flow 0.6 ml/min; retention time 1.99 min; detection at 214 and 254 nm). Further characterisation was carried out using mass spectrometry, giving m/z value 1050.4 [MH²⁺].

### Example 7

### Plasma Stability of Compounds 1 to 8

Mouse plasma (non-sterile) was purchased from Rockland, Pa., USA. This plasma is stabilized with heparin, sodium. The substance was dissolved in PBS and plasma, respectively, at concentrations 0.1/0.2 mg/mL. Both blank samples (solvent without peptide) and peptide dissolved in plasma/PBS were incubated at 37° C. for about 4 hours. After incubation the proteins were removed by ultrafiltration using non-sterile Ultrafree®-MC centrifuge tubes with filter insert from Millipore Co. (Amicon). The cut-off of the filters was 30,000 NMWL Prior to centrifugation the plasma samples were diluted 1:1 with water. The samples were analysed by HPLC using visible detection.

The substance was dissolved in PBS, concentration 0.1 mg/mL. The fluorescence intensity of the ultracentrifuged samples was measured using Fluoroskan Ascent® FL equipped with plate reader (Thermo Labsystems Oy, Finland). Excitation wavelength was at 646 nm and emission wavelength at 678 nm and measurements were performed at two different concentrations of the substance, 6.5 μg/mL and 23 μg/mL plasma.

The Ultimate 3000 micro liquid chromatograph equipped with UV-Vis detector was applied in this study. The solution has an intense bluish colour and absorbs well at 650 nm.

The chromatography was performed on an X-Terra RP18 column 2.1×150 mm, 3.5 μm particles from Waters using a gradient elution of acetonitrile (ACN) and phosphate buffer (20 mM, pH 7.1); 650 nm detection; flow rate 0.1 mL/min; injection volume: 5 μL. Gradient: initiated at 22% ACN in buffer, increasing linearly to 50% ACN over 12 min; rapid linear increase of gradient to 90% ACN for 2 min followed; then equilibration to the starting mixture. Total analysis time was 20 min, with a retention time for the main peak of ˜9 min. Degradation/impurities were reported as changes in purity of the main peak. The results are shown in Table 4:

TABLE 4
Plasma stability of Compounds 1 to 8.

| | % main peak area |
|---|---|
| Compound | (after 4 h incubation in mouse plasma) |
| 1 | Not measured. |
| 2 | Not measured. |
| 3 | Not measured. |
| 4 | 100 |
| 5 | 46 |
| 6 | 100 |
| 7 | 70 |
| 8 | 93 |

### Example 8

### Fluorescence Polarisation Plasma Protein Binding Assay of Compounds 1 to 8

Compounds 1 to 8 were tested in a fluorescence polarisation protein binding assay using human plasma and assay buffer (PBS/0.05% Tween). 40 μl peptide (˜5 μM) was incubated in 40 μl PBS or human plasma. Fluorescence polarisation was measured in a Tecan Safire plate reader (Ex635/Em678) and data is reported as % increase in polarisation value when adding plasma.

The results are shown in Table 5:

TABLE 5
Polarisation values summary (Ex635 nm, Em678 nm,
~2.5 μM substance) for Compounds 1 to 8.

| | Assay | | Increase in |
|---|---|---|---|
| | buffer | Plasma | polarisation |
| Compound | (mP) | (mP) | values (%) |
| 1 | 184 | 218 | 18 |
| 2 | 178 | 217 | 21 |
| 3 | 219 | 298 | 36 |
| 4 | 183 | 209 | 14 |
| 5 | 187 | 227 | 22 |
| 6 | 178 | 199 | 12 |
| 7 | 179 | 232 | 29 |
| 8 | 184 | 215 | 17 |

### Example 9

### Collagen Binding Assay of Compounds 1 to 8

Commercially available collagen-covered 96-well plates were used (BD Biocoat, Art. code BDAA 356649, Becton, Dickinson Biosciences, Two Oak Park, Bedford, Mass. 01730). Triplicate wells were made of each test Compound at 30 nM, 100 nM or 300 nM (1000 nM included in some cases), and the plate was incubated in the plate reader for one hour at 37° C. with shaking every other minute*. The volume in each well was 200 μl. At the end of the incubation, 150 μl supernatant was transferred to an untreated 96-well plate, and the fluorescence was read with excitation at 646 nm and emission at 678 nm wavelengths.

*Compound 3 was incubated at 37° C. in a heating cabinet with shaking every 5 minutes (microplate with a lid).

For calculation of the degree of binding, the fluorescence from the supernatants was compared to the fluorescence from 150 μl aliquots with the same initial concentrations. In all cases, median values from 3 wells were used. The results are shown in Table 6:

TABLE 6
Collagen binding assay (% bound), Ex646/Em678 nm.

| Initial | | | | | | | | |
|---|---|---|---|---|---|---|---|---|
| conc. | Compound Number | | | | | | | |
| (nM) | 1 | 2 | 3 | 4 | 5 | 6 | 7 | 8 |
| 30 | 94.3 | 87 | 80.9 | 23.7 | 83.6 | 15.7 | 89.7 | 92.1 |
| 100 | 71.2 | 81.1 | 84.6 | 13.9 | 96.1 | 9.4 | 87.2 | 85.9 |
| 300 | 70.5 | 83 | 77.2 | 9.6 | 50.9 | 12.4 | 39.7 | 56.7 |
| 1000 | np | np | 40.3 | np | 30.4 | np | 20.0 | 32.7 |

np = not performed.

### Example 10

### Competition Assay for Compounds 1 to 8

A classical competition assay using ¹²⁵I-echistatin was performed in order to check the affinity (Kᵢ) of the RGD-Cy^{D} conjugates (Compounds 1 to 8) towards membranes expressing. the α_{V}β₃ receptor. Kᵢ was determined in receptor competition studies with membranes prepared from human endothelial cells. Membranes from the human endothelial adenocarcinoma cell line EA-Hy926 that express several integrins including α_{V}β₃ were prepared and used as a receptor source. Competitive binding of ¹²⁵I-Echistatin, a known substrate for several integrins including α_{V}β₃, was carried out with varying concentrations of cold compounds.

The results are shown in Table 7:

TABLE 7
Ki measurements (competition assay using ¹²⁵I-Echistatin).

| Compound | Ki (nM) |
|---|---|
| 1 | 1.9 |
| 2 | — |
| 3 | 2.6 |
| 4 | 1.8 |
| 5 | 1.7 |
| 6 | 1.6 |
| 7 | 7.4 |
| 8 | 2.9 |

### Example 11

### In Vivo testing of Compounds 1 to 8

(a) Animal Model.

Female BALB c/A nude (Bom) mice were used in the study. The use of the animals was approved by the local ethics committee. As the animals were immunocompromised, they were housed in individually ventilated cages (IVC, Scanbur BK) supplied with HEPA filtered air. The animals had ad libitum access to “Rat and Mouse nr. 3 Breeding” diet (Scanbur BK) and tap water acidified by addition of HCl to a molar concentration of 1 mM (pH 3.0). In order to protect the animals during handling and all procedures before the imaging procedure, they were handled under conditions of laminar HEPA filtered air.

The animals were allowed an acclimatisation period of at least 5 days before being injected s.c. with HT-29 tumour cell suspensions at two sites (shoulder and left, lower flank) with a nominal dose of 2.5-3×10⁶ cells per injection in a volume of 100 μl. The s.c. injections were performed under light gas anaesthesia. The tumours were allowed to grow for 2-4 weeks.

For immobilisation during the optical imaging procedure, the animals were anaesthetized in a coaxial open mask to light surgical level anaesthesia with Isoflurane (typically 1.5-2%) with oxygen as the carrier gas. The animals were supplied external heating from a heating blanket to sustain normal body temperature for the duration of the imaging (up to 3 hours). A Venflon catheter was placed in the tail vein for contrast agent administration. Each animal was given one contrast agent injection.

To avoid artefacts from imaging probe in the skin, a ˜3 mm diameter piece of skin over tumour and muscle was removed before imaging, but while the animal was anaesthetized. The animals were sacrificed by cervical dislocation at the end of the experiment.

(b) Imaging Protocol.

The laser was turned on at least 15 minutes before the start of the experiment for the output to stabilise. A small stack of white printer paper was imaged to obtain a flatfield image which was used to correct for illumination inhomogeneities. For the kinetics imaging the animals were placed inside the imaging dark box on a heating blanket (BioVet) with a temperature of 40° C. Respiration and temperature were used to monitor the anaesthesia depth during imaging. The animals were imaged one at a time. Pre injection images with the laser light source and with a white light source were taken of all the animals. The emission filters were in place for both light sources, effectively making the white light image an image with illumination at the receive frequencies.

The test substance was injected iv through the Venflon and was followed by a 0.2 ml saline flush. A time series of images were taken from the beginning of the injection with one new image every 30 seconds. The images were stored locally before being transferred to a server.

Image analysis was performed with custom written MATLAB software. Regions of interest were drawn around the part of the tumour and muscle not covered by skin. A third region was placed over a part of the skin where there was no tumour or kidney tissue underneath to compromise the signal. The mean signal of the pixel values inside each region was calculated. The mean signal and pixel standard deviation was calculated.

(c) Results.

Based on the in-vitro data from example 7-10, in-vivo results from compounds 1, 2, 4 and 6 are presented. Tumour enhancement is quantified by a target to background ratio (TBR) defined as the ratio of the mean tumour region intensity divided by the mean muscle region intensity.

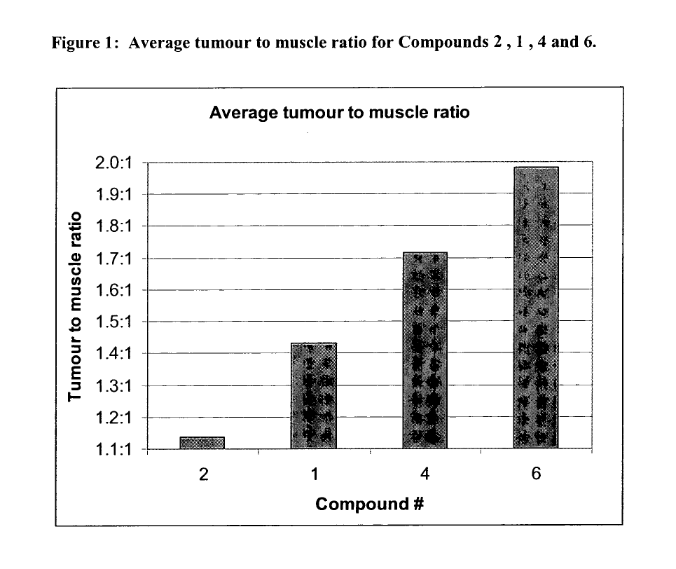

Compound 2 (neg-RGD scrambled peptide) gave a TBR of 1.14. Compound 1 [Cy5(2)-RGD] gave a ratio of 1.43. Compounds 4 and 6 show the expected improvement with ratios of 1.72 and 1.98 respectively. The results are shown in FIG. 1.

What is claimed is:

1. An imaging agent suitable for in vivo optical imaging of the mammalian body which comprises a conjugate of Formula I:
[BTM]-(L)ₙ-Cy^{D} (I)
where:
BTM is a synthetic biological targeting moiety;
Cy^{D} is a cyanine dye of Formula II: 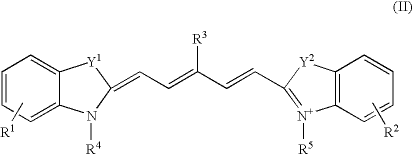
where:
Y¹ and Y² are independently —O—, —S—, —NR⁶— or —CR⁷R⁸— and are chosen such that at least one of Y¹ and Y² is —CR⁷R⁸—;
R¹ and R² are independently H, —SO₃M¹ or R^{a}, where M¹ is H or B^{c}, and B^{c} is a biocompatible cation;
R³ is H, C₁₋₅ alkyl, C₁₋₆ carboxyalkyl or an R^{a} group;
R⁴ to R⁶ are independently C₁₋₅ alkyl, C₁₋₆ carboxyalkyl or R^{a};
R⁷ is H or C₁₋₃ alkyl;
R⁸ is R^{a} or C₁₋₆ carboxyalkyl;
R^{a} is C₁₋₄ sulfoalkyl;
L is a synthetic linker group of formula -(A)ₘ- wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1;
with the provisos that:
(i) the cyanine dye comprises at least one R^{a} group and a total of 3 to 6 sulfonic acid substituents from the R¹, R² and R^{a} groups;
(ii) the imaging agent does not comprise a fluorescence quencher;
(iii) when the BTM is a peptide, at least one of the termini of the peptide has conjugated thereto a metabolism inhibiting group (M^{IG}) where M^{IG} is a biocompatible group which inhibits or suppresses enzyme metabolism of the BTM peptide.

2. The imaging agent of claim 1, where R³ is H.

3. The imaging agent of claim 1, where Y¹ and Y² are each independently —CR⁷R⁸—.

4. The imaging agent of claim 3, where R⁷ is CH₃.

5. The imaging agent of claim 1, where Cy^{D} has a total of 4 sulfonic acid substituents chosen from the R¹, R² and R^{a} groups.

6. The imaging agent of claim 1 where the R^{a} groups are independently of formula —(CH₂)ₖSO₃M¹, where M¹ is as defined in claim 1, and k is an integer of value 1 to 4.

7. The imaging agent of claim 6, where k is 3 or 4.

8. The imaging agent of claim 1, where R¹═R²═SO₃M¹.

9. The imaging agent of claim 8, where the SO₃M¹ substituents are at the 5-position of the indole/indolenine rings.

10. The imaging agent of claim 1, where Cy^{D} is of Formula III: 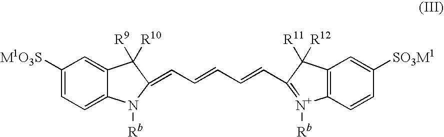
where:
R^{b} is independently an R^{a} group or C₁₋₆ carboxyalkyl;
R⁹ to R¹² are independently C₁₋₅ alkyl or an R^{b} group, and are chosen such that either R⁹═R¹⁰═R^{c} or R¹¹═R¹²═R^{c}, where R^{c} is C₁₋₂ alkyl;
R^{a} and M¹ are as defined in claim 1.

11. The imaging agent of claim 1, where BTM is chosen from:
(i) a 3-100 mer peptide;
(ii) an enzyme substrate, enzyme antagonist or enzyme inhibitor;
(iii) a receptor-binding compound;
(iv) an oligonucleotide;
(v) an oligo-DNA or oligo-RNA fragment.

12. The imaging agent of claim 11, where BTM is a 3-100 mer peptide.

13. The imaging agent of claim 12, which is of Formulae IVa or IVb:
[Cy^{D}]-(L)ₙ-[BTM]-Z² (IVa);
Z¹-[BTM]-(L)ₙ-[Cy^{D}] (IVb);
where:
Z¹ is attached to the N-terminus of the BTM peptide, and is H or M^{IG};
Z² is attached to the C-terminus of the BTM peptide and is OH, OB^{c}, or M^{IG},
where B^{c} is a biocompatible cation and M^{IG} is a biocompatible group which inhibits or suppresses enzyme metabolism of the BTM peptide.

14. The imaging agent of claim 13, where Z¹═Z²=M^{IG}.

15. A pharmaceutical composition which comprises the imaging agent of claim 1 together with a biocompatible carrier, in a form suitable for mammalian administration.

16. The pharmaceutical composition of claim 15, which has a dosage suitable for a single patient and is provided in a suitable syringe or container.

17. A kit for the preparation of the pharmaceutical composition which comprises an imaging agent of claim 1 together with a biocompatible carrier, in a form suitable for mammalian administration, which comprises said imaging agent in sterile, solid form such that upon reconstitution with a sterile supply of the biocompatible carrier, dissolution occurs to give the desired pharmaceutical composition.

18. The kit of claim 17, where the sterile, solid form is a lyophilised solid.

19. A conjugate of Formula Ia:
[BTM]-(L)ₙ-Cy^{D} (Ia)
where: BTM, L and n are as defined in claim 1, and Cy^{D} is of Formula IIIa: 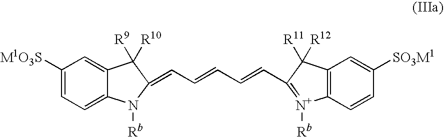
where:
R⁹ to R¹² are independently R^{b} or R^{c} groups, and are chosen such that
one of R⁹ to R¹² is an R^{a} group, and the others are each R^{c} groups, where R^{c} is C₁₋₂ alkyl;
R^{a} is C₁₋₄ sulfoalkyl, R^{b} is independently an R^{a} group or C₁₋₆ carboxyalkyl and M¹ is M¹ is H or B^{c},
and B^{c} is a biocompatible cation.

20. A cyanine dye of Formula IIIa as defined in claim 19, useful in the preparation of the conjugate of claim 19.

21. The cyanine dye of claim 20, which further comprises a group Q^{a}, where Q^{a} is a reactive functional group suitable for conjugation to a BTM.

22. The cyanine dye of claim 20, where R^{b} is independently
—(CH₂)ₖSO₃M¹, where k is an integer of value 1 to 4.

23. A method of in vivo optical imaging of the mammalian body which comprises use of either the imaging agent of claim 1 or the pharmaceutical composition which comprises the imaging agent of claim 1 together with a biocompatible carrier, in a form suitable for mammalian administration to obtain images of sites of localisation of the BTM in vivo.

24. The method of in vivo optical imaging of the mammalian body which comprises use of either the imaging agent of claim 1, where the imaging agent of claim 1 or the pharmaceutical composition which comprises the imaging agent of claim 1 together with a biocompatible carrier, in a form suitable for mammalian administration which has been previously administered to said mammalian body.

25. The method of claim 24, which comprises the steps of:
(i) a tissue surface of interest within the mammalian body is illuminated with an excitation light;
(ii) fluorescence from the imaging agent, which is generated by excitation of the Cy^{D} is detected using a fluorescence detector;
(iii) the light detected by the fluorescence detector is optionally filtered to separate out the fluorescence component;
(iv) an image of said tissue surface of interest is formed from the fluorescent light of steps (ii) or (iii).

26. The method of claim 25 where the excitation light of step (i) is continuous wave (CW) in nature.

27. The method of claim 24 which comprises:
(a) exposing light-scattering biologic tissue of said mammalian body having a heterogeneous composition to light from a light source with a pre-determined time varying intensity to excite the imaging agent, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

28. The method of claim 23, where the optical imaging method comprises fluorescence endoscopy.

29. The method of claim 23, where the in vivo optical imaging is used to assist in the detection, staging, diagnosis, monitoring of disease progression or monitoring of treatment of a disease state of the mammalian body.

30. A method of detection, staging, diagnosis, monitoring of disease progression or monitoring of treatment of a disease state of the mammalian body which comprises the in vivo optical imaging method of claim 23.
